(19) United States
(12) Patent Application Publication
(10) Pub. No.: US 2002/0103498 A1
(43) Pub. Date: Aug. 1, 2002

(54) Flexible endoscopic grasping and cutting device and positioning tool assembly

(75) Inventor: Anthony C. Pagedas, Greendale, WI (US)

Correspondence Address: RYAN KROMHOLZ & MANION, S.C., POST OFFICE BOX 26618, MILWAUKEE, WI, 53226, US

(73) Assignee: Ancel Surgical R&D, Inc.

(21) Appl. No.: 09/775,015

(22) Filed: Feb. 1, 2001

(51) Int. Cl. A61B 17/32

(52) U.S. Cl. 606/170; 606/205

(57) ABSTRACT

An endoscopic, laparoscopic or mini-laparoscopic assembly including an elongate instrument having a flexible shaft, and a positioning mechanism capable of engaging and positioning the elongate instrument within a body cavity.

### FIELD OF THE INVENTION

The present invention relates to internal surgery and, more specifically, to a flexible endoscopic grasping and cutting device to be used with a novel positioning tool during such surgery. The device is for use in accessing and cutting tissue or other masses in a body cavity. The device may be further used for removal of matter, such as polyps, or may be used in coagulation procedures.

### BACKGROUND OF THE INVENTION

In recent years, the applications for laparoscopic and endoscopic surgery have expanded to include many different procedures. A benefit of this type of operation is the relatively quick recovery period experienced by patients, due to the small incisions that are made in the body. These incisions reduce the trauma and the required healing compared to traditional surgery. Laparoscopic tubes and sleeves with diameters on the order of 10 millimeters are inserted in the body cavity. Various instruments and a video camera are typically directed through laparoscopic sleeves for performing and monitoring the surgical steps. Recently, mini-laparoscopic procedures have become feasible as well. Mini-laparoscopic procedures allow the surgeon to employ much smaller ports, some as small as 3 millimeters in diameter. These smaller ports ensure a more comfortable procedure for the patient with shorter recovery periods.

A particular concern in laparoscopic or mini-laparoscopic surgery is the accuracy in cutting tissues and other masses during a surgery. Typically, the port through which the various surgical tools are inserted in the body cavity is relatively rigid and inflexible along its length. Further, the tools inserted, such as grasping and cutting tools, are also relatively inflexible. This common feature limits the maneuvering capabilities of the surgeon during surgical procedures, thereby causing difficulty in reaching tissue and other mass not directly in the linear path of the rigid tool in use. Often multiple ports must be inserted in the body cavity through which an additional tools may be inserted. These additional tools may be similar to the first inserted tool, or have different functions, such as cutting, grasping, or morcellating. Further, positioning tools may be inserted, and are used to aid in maneuvering the tools and to conduct procedures not within the functional range of other tools. These positioning tools allow the surgeon to access an area not initially within reach of the functional tools. However, all of these tools are typically rigid and are difficult to maneuver in the body cavity. Further, the rigidity of the various surgical tools allows little positional variation despite the use of a positioning tool.

For the foregoing reasons, there is a need for an endoscopic, laparoscopic or mini-laparoscopic surgical assembly, and a method of using such an assembly, that allows efficient access of various surgical sites for cutting or removal of tissue or other mass from a body cavity, or cauterization of a site within a body cavity.

### SUMMARY OF THE INVENTION

According to the present invention, the foregoing and following objects and advantages are achieved by providing an endoscopic, laparoscopic or mini-laparoscopic assembly for accessing various operating sites in a single body cavity, and also a method for using the assembly. An assembly having the features of the present invention comprises an elongate operating instrument and an elongate positioning mechanism. The operating instrument may have a manually operable actuating end, although it is within the scope of this invention to include non-manual activation. The elongate operating instrument of this invention further includes an operative end. The operative end may include at least one work member known in the art, such as, but not limited to, grasping means and cutting means. The operative end may also include a plurality of work members, as in a combination grasper and cutter with a cauterization tip. The elongate operating instrument further includes a flexible, elongate shaft for connecting the actuating end to the operating end. The flexible shaft allows the operating instrument to be easily positioned within a body cavity by allowing the surgeon to temporarily flex or bend the shaft to reach a particular location within the body cavity at a particular angle. The operating instrument may be provided with an optional electrode post which is used to heat the tip of the operative end for instant cauterization.

The endoscopic or laparoscopic assembly of the present invention further includes an elongate positioning mechanism or mechanical guide for positioning the elongate operating tool. The elongate positioning mechanism preferably includes a grasping mechanism for grasping and positioning the operating tool. The grasping mechanism may be of any suitable means able to releasably receive and thereby position the operating tool, however the preferred grasping means includes an openable encircling element. Alternatively, the grasping means may be a forked member. It is also within the scope of this invention to include an elongate positioning mechanism which includes an engaging end rather than a grasping means.

It is an object of the present invention to provide an endoscopic, laparoscopic or mini-laparoscopic assembly that may be accurately manipulated and controlled in a single body cavity yet access several sites within that cavity.

Another object of the invention is to provide a surgical assembly that includes an elongate, operating instrument having a flexible shaft wherein the flexible shaft may be engaged by a cooperating positioning mechanism to position the operating end of the elongate, operating instrument at multiple sites in a body cavity.

Another object of the invention is to provide a positioning mechanism that includes means for engaging the flexible shaft of the elongate, operating instrument.

Methods of using the assembly comprise the steps of directing the elongate, operating instrument and positioning mechanism into a body cavity, engaging the flexible shaft of the operating instrument with the distal end of the positioning instrument, positioning the operating end of the elongate, operating instrument with the positioning mechanism to a position adjacent a work site in the cavity, manipulating the actuator end to cause the operating end to do work at the selected surgical site, and disengaging the distal end of the positioning mechanism from the flexible shaft. Additional steps may include positioning the operating end at a plurality of work sites, removing the assembly from the body cavity, sterilizing the elongate, operating instrument and/or the positioning mechanism for reuse.

Accordingly, the present invention provides an economical means for achieving greater efficiency, control and safety in positioning an operating tool within a body cavity during laparoscopic surgery.

These and other features, aspects and advantages of the present invention will become better understood with regard to the following description. Other objects, advantages and novel features of the invention will become apparent to those skilled in the art. As will be realized, the invention is capable of other and different embodiments and use in other applications, and its several details are capable of modification in various obvious respects, all without departing from the invention. Accordingly, the drawings and descriptions are to be regarded as illustrative in nature and not restrictive.

### DESCRIPTION OF THE DRAWINGS

FIG. 1 is a perspective view of the assembly of the present invention and showing an encircling guide element cooperating with the elongate instrument flexible shaft.

FIG. 2 is an enlarged, fragmentary perspective view of the operative end of the elongate instrument of the assembly of the present invention and showing a cutting blade and grasper.

FIG. 3 is an enlarged, fragmentary perspective view of a positioning mechanism of the assembly of the present invention and showing the encircling portion in the open position.

FIG. 3a is an enlarged, fragmentary perspective view of positioning mechanism shown in FIG. 3 but showing the encircling portion in the closed position.

FIG. 4 is a view of one embodiment of the assembly with each element directed through a respective laparoscopic port into a body cavity and showing flexible positioning of the assembly in dashed line.

FIG. 5 is a perspective view of the assembly of the present invention and showing an alternative embodiment forked guide element cooperating with the elongate instrument flexible shaft.

FIG. 6 is a view of the assembly shown in FIG. 5 with each element directed through a respective laparoscopic sleeve into a body cavity and showing the forked positioning mechanism moving the flexible shaft of the elongate instrument.

FIG. 7 is a view of an alternative embodiment assembly in a body cavity and showing an alternative embodiment guide element cooperating with the elongate instrument flexible shaft.

### DETAILED DESCRIPTION

Although the disclosure hereof is detailed and exact to enable those skilled in the art to practice the invention, the physical embodiments herein disclosed merely exemplify the invention which may be embodied in other specific structure. While the preferred embodiment has been described, the details may be changed without departing from the invention, which is defined by the claims.

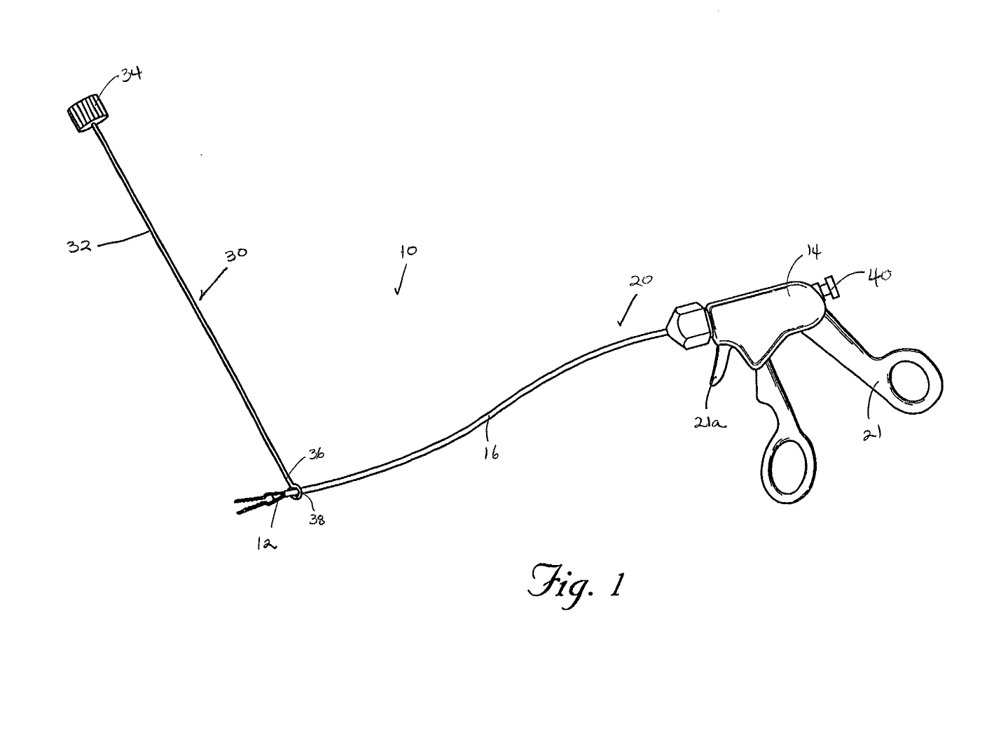

As shown in FIG. 1, the preferred embodiment of the endoscopic or laparoscopic assembly 10 of the present invention includes an elongate instrument 20 and an elongate positioning mechanism 30. The elongate instrument 20 includes an operative end 12, an actuator end 14, and a flexible shaft portion 16 located intermediate the operative end 12 and the actuator end 14. The positioning mechanism 30 is used for positioning the elongate instrument 20 and its operating end 12 adjacent predetermined operation sites (not seen in this view). The positioning mechanism 30 preferably includes an elongated rod 32 that includes a handle portion 34 having a distal end 36. As seen particularly in the views of FIGS. 1, 3 and 3a, the distal end 36 of the positioning mechanism may include grasping means, seen as an openable encircling element 38. As illustrated in FIGS. 3 and 3a, the encircling element 38 may be alternated between opened and closed positions by rotation of the handle portion 34. Optionally, embodiments of the elongate instrument 20 may also include an electrode post 40 at the actuator end 14 of the elongate instrument 20. The optional electrode post 40 may be used to heat the tip 41 thereby providing cauterization at the surgical site.

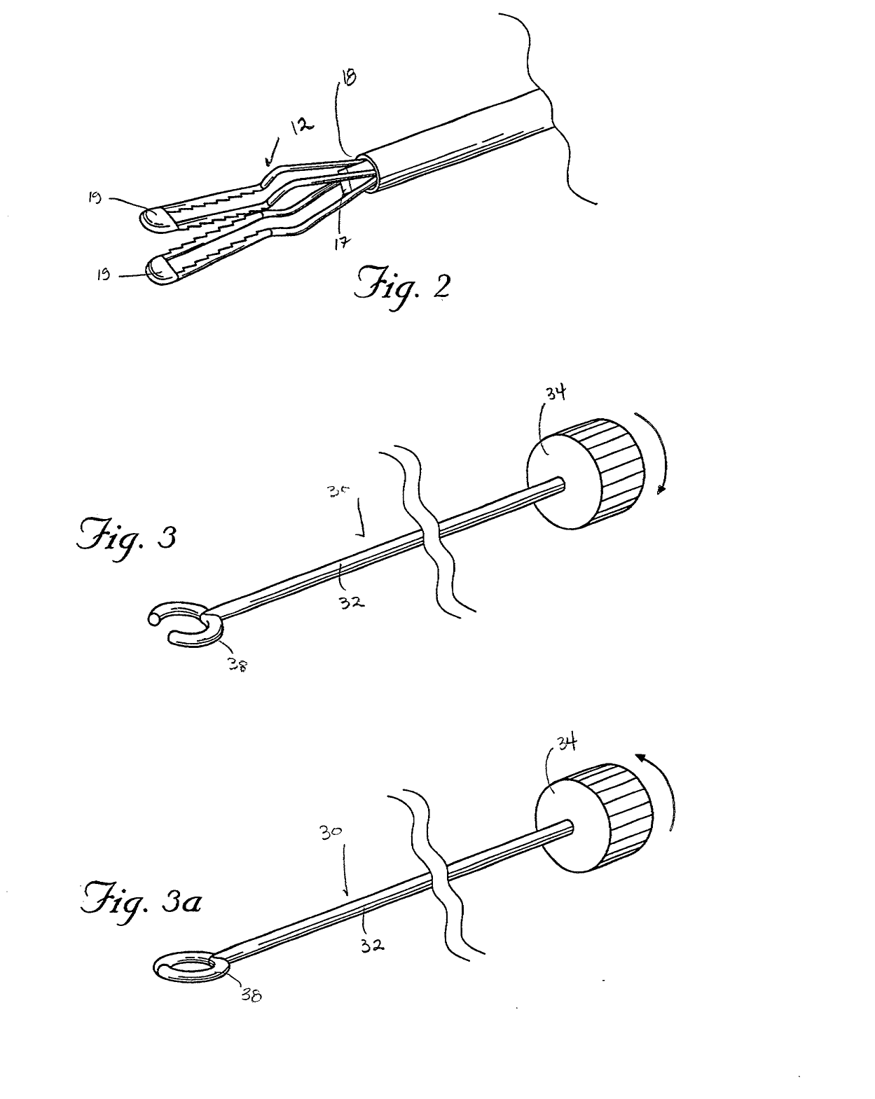

As seen in FIG. 2, the operative end 12 of the elongate instrument 20 may include any tool desired by the user, however the combination grasper and cutter end 18 shown in FIG. 2 is presently believed to have the best practical application. This configuration includes grasping members 19 and cutting edge 17. The operative end is manipulated by scissor-like handles 21 and trigger 21a seen in FIG. 1. Use of a combination operative end 12 allows the surgeon to perform multiple tasks with a single tool.

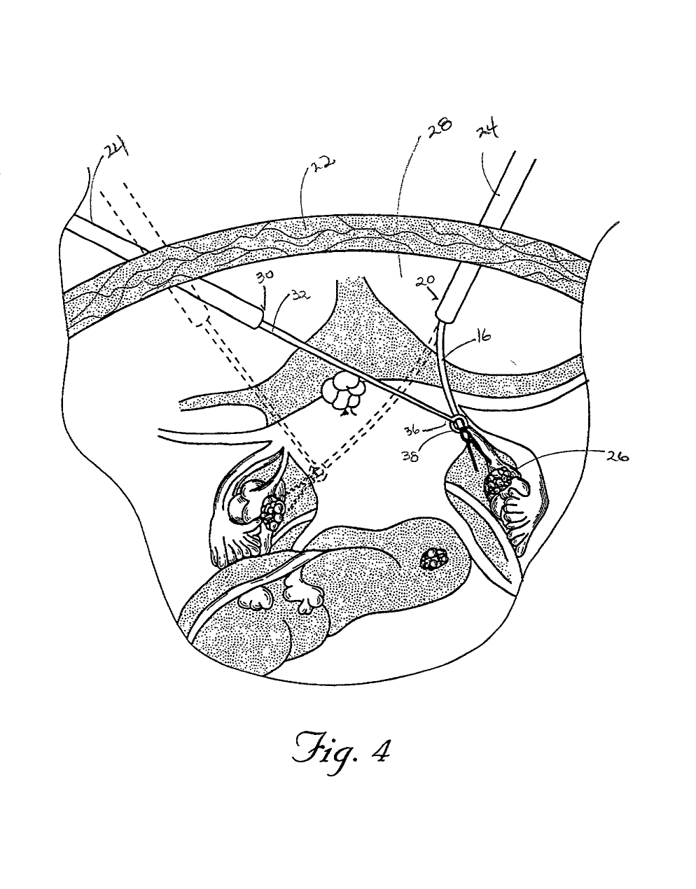

As seen in the view of FIG. 4, the assembly 10 comprising elements 20 and 30 of the present invention, allows for facile access to multiple operating sites within a body cavity 28 without the need for multiple insertion sites. The elongate instrument 20 and positioning mechanism 30 are inserted through the abdominal wall 22 and into a cavity 28 through respective laparoscopic ports 24, while the actuator end 14 and handle portion 34 remain outside the body cavity and accessible by the surgeon. As illustrated in FIG. 4, the distal end 36, seen as an encircling element 38 in this view, of the positioning mechanism 30 engages the flexible shaft 16 of the elongate instrument 20, thereby directing the flexible shaft 16 and operative end 12 to the desired position, seen as adjacent a mass 26 in this view.

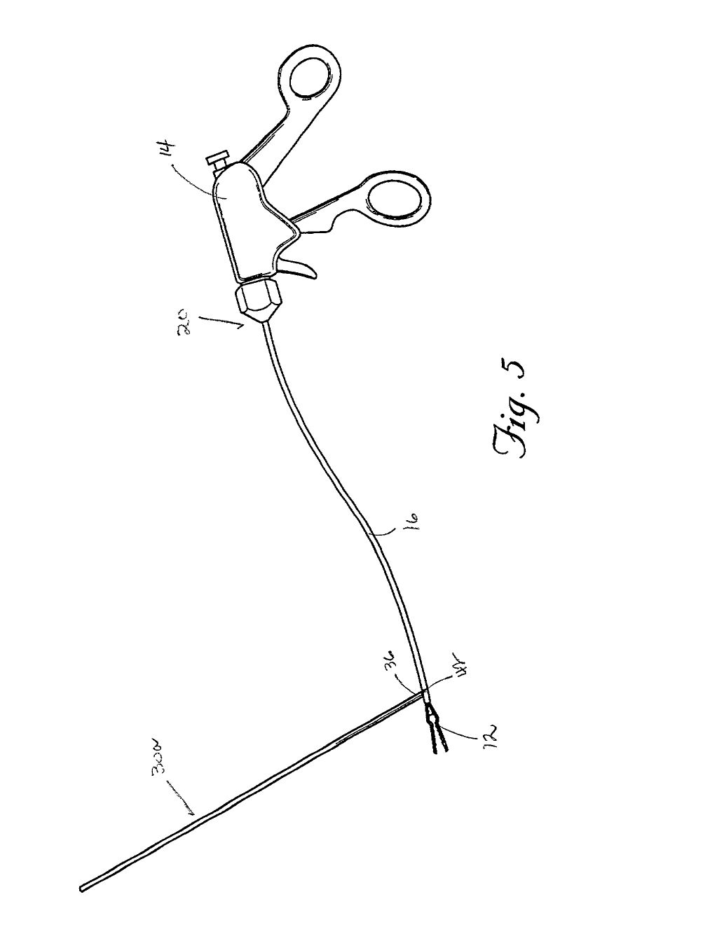

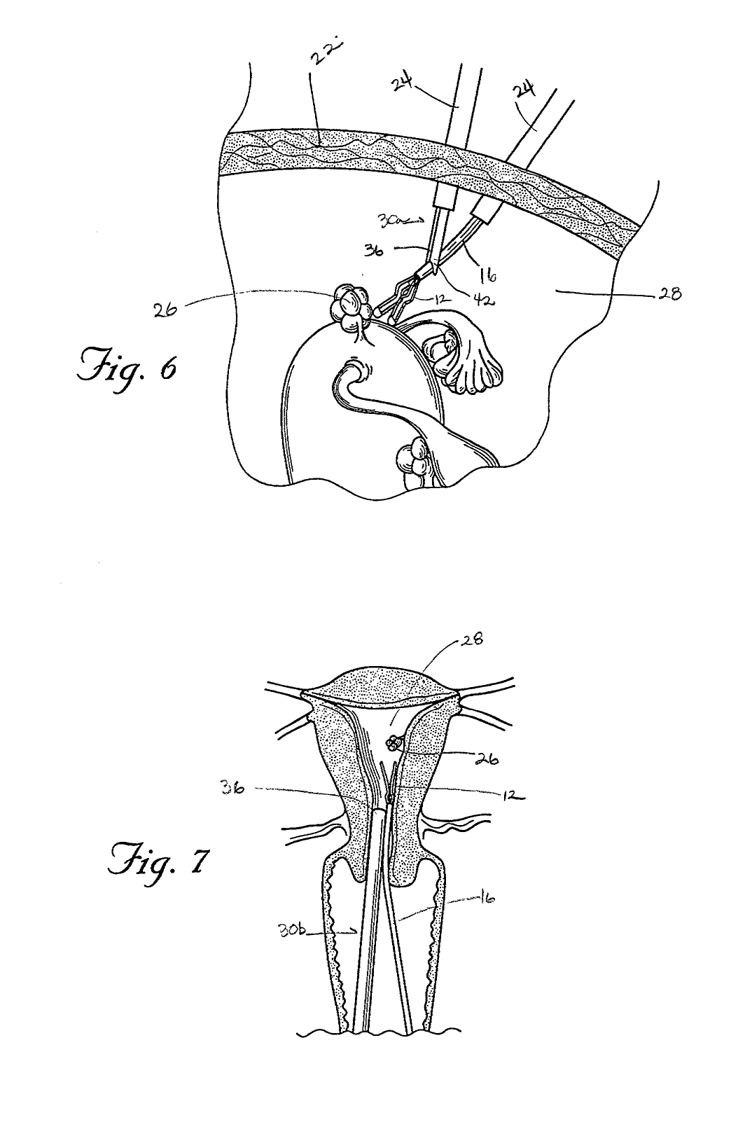

FIGS. 5 and 6 illustrate another embodiment of the laparoscopic assembly 10. As seen in these views, an alternative positioning mechanism 30a includes a distal end 36 having a forked portion 42 including at least two prongs 43. The forked portion 42 may engage the flexible shaft 16 between the prongs 43, of the elongate instrument 20 and thereby position the operative end 12 relative a mass 26 or other surgical site. As in the previously described embodiment, the elongate instrument 20 and alternative positioning mechanism 30a are each directed into a body cavity 28, preferably through respective laparoscopic sleeves 24, so that the actuator end 14 is accessible by the surgeon. As seen particularly in FIG. 6, the operative end 12 is positioned by engaging the flexible shaft 16 between the prongs 43 of the forked portion 42 of the positioning mechanism 30. The operative end 12 may be further positioned relative multiple surgical sites.

A further alternative embodiment of the assembly 10 may be viewed in FIG. 7. As depicted in this view an alternative positioning mechanism 30b is seen slidably engaging the flexible shaft 16 thereby positioning the operative end 12 relative to a mass 26. In this embodiment, the positioning mechanism 30b is pressed against the flexible shaft 16 to assist in placement of the end 12 for operative removal of a mass 26.

Methods of using the assemblies 10 shown in FIGS. 1-7 include the steps of: directing the elongate, operating instrument 20 and positioning mechanism 30 into a body cavity 28; engaging the flexible shaft 16 of the elongate, operating instrument 20 with the distal end 36 of the positioning mechanism 30; positioning the operating end 12 of the elongate instrument 20 with the positioning mechanism 30 to a position adjacent a work site, such as a mass 26 in the body cavity 28; manipulating the actuator end 14 of the elongate instrument 20 to activate the operating end 12 to do work at the selected work site; and disengaging the distal end 36 of the positioning mechanism 30 from the flexible shaft 16. Additional steps may include positioning the operating end 12 at a plurality of work sites; removing the assembly 10 from the body cavity 28; and sterilizing the elongate operating instrument 20 and/or the positioning mechanism 30 for reuse.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

What is claimed is:

1. An endoscopic or laparoscopic assembly comprising:
an elongate instrument having an operative end and a manually operative actuator end; and
an elongate positioning mechanism for positioning said elongate instrument.

2. The assembly of claim 1 wherein said elongate instrument includes a shaft having a flexible portion.

3. The assembly of claim 1 wherein said operative end includes a cutting blade.

4. The assembly of claim 3 wherein said operative end further includes grasping means.

5. The assembly of claim 2 wherein said positioning mechanism includes grasping means.

6. The assembly of claim 4 wherein said grasping means comprises an encircling guide element arranged to receive said shaft of said elongate instrument.

7. The assembly of claim 6 wherein said guide element is arranged to releasably receive said shaft.

8. The assembly of claim 6 wherein said guide element is arranged to be alternatively opened and closed relative to said shaft whereby said shaft may be moved to a predetermined position.

9. The assembly of claim 5 wherein said grasping means comprises a forked element arranged to receive and guide said shaft to a predetermined position.

10. An endoscopic or laparoscopic assembly comprising:
an elongate instrument having an operative end and a manually operative actuator end; and
an elongate positioning mechanism capable of removably engaging and positioning said elongate instrument.

11. The assembly of claim 10 wherein said elongate instrument includes a shaft having a flexible portion.

12. The assembly of claim 10 wherein said operative end includes a cutting blade.

13. The assembly of claim 12 wherein said operative end further includes grasping means.

14. The assembly of claim 11 wherein said positioning mechanism includes grasping means.

15. The assembly of claim 13 wherein said grasping means comprises an encircling guide element arranged to receive said shaft of said elongate instrument.

16. The assembly of claim 15 wherein said guide element is arranged to releasably receive said shaft.

17. The assembly of claim 15 wherein said guide element is arranged to be alternatively opened and closed relative to said shaft whereby said shaft may be moved to a predetermined position.

18. The assembly of claim 14 wherein said grasping means comprises a forked element arranged to receive and guide said shaft to a predetermined position.

19. A surgical method for manipulating an endoscopic or laparoscopic assembly in a body cavity, said method comprising:
directing an endoscopic or laparoscopic assembly into a body cavity, said assembly including:
an elongate operating instrument having a flexible shaft, said flexible shaft including an operative end and an actuator end said actuator end arranged to activate said operative end;
a positioning mechanism including a distal end; said distal end including means to releasably engage said flexible shaft;
engaging the flexible shaft of said elongate instrument with said distal end of said positioning mechanism;
positioning said flexible shaft and said operative end with said engaged positioning mechanism to a position adjacent a pre-selected surgical site;
actuating the operative end of the elongate instrument by manipulating the actuator end so that said operative end performs work at said preselected surgical site;
disengaging the distal end of the positioning mechanism from the flexible shaft; and
removing the assembly from the body cavity.
